(19) United States
(12) Patent Application Publication
(10) Pub. No.: US 2006/0167062 A1
(43) Pub. Date: Jul. 27, 2006

(54) Enema preparation

(76) Inventors: Takashi Maeda, Tokushima (JP); Hisashi Nagamoto, Tokushima (JP); Masatoshi Chihiro, Tokushima (JP)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 10/564,743

(22) PCT Filed: Jul. 16, 2004
(86) PCT No.: PCT/JP04/10546

§ 371 Date: Jan. 17, 2006

(30) Foreign Application Priority Data
Jul. 17, 2003 (JP) 2003-275955

(51) Int. Cl. A61K 31/4439

(52) U.S. Cl. 514/340

(57) ABSTRACT

The object of this invention is to provide an enema preparation effective in treating inflammatory bowel diseases. The enema preparation of this invention includes at least one selected from the group consisting of thiazole compounds having the following general formula and the salts thereof: 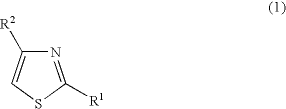
wherein R¹ represents a phenyl group which may have 1 to 3 lower alkoxy groups as substituents on the phenyl ring and R² a pyridyl group which may have 1 to 3 carboxyl groups as substituents on the pyridine ring.

### TECHNICAL FIELD

The present invention relates to an enema preparation.

### BACKGROUND ART

Mesalazine, prednisolone, methylprednisolone, dexamethasone, betamethasone and hydrocortisone are known as pharmacologically active ingredients of marketed drugs for treating regional enteritis or ulcerative colitis. These drugs are administered in the form of an oral preparation such as tablet, pill, solution, suspension or capsule, injection, suppository or enema preparation.

These dosage forms are, however, not expected to produce satisfactory therapeutic effects.

For example, in oral administration using oral preparations (tablets, pills, solutions, suspensions and capsules), or intravenous, intramascular, intradermal, hypodermic or intraperitoneal administration using injections, pharmacologically active ingredients are not only delivered to the affected region, but dispersed widely throughout the body, and therefore their rapid action is not expected. Moreover, to allow them to develop desired therapeutic effects, it becomes necessary to increase the amount of pharmacologically active ingredients administered. However, the increase of the amount administered may give rise to a problem of causing the onset of adverse reactions.

In administration using suppositories, their rapid action can be expected because the pharmacologically active ingredients rapidly reach the affected region needing treatment. They still have disadvantages, however, in that: (1) They can be applied to the affected region only around the rectum; and (2) They are easily ejected through anus and they can stay in the affected region for a short period, and therefore sufficient therapeutic effects may not be exerted.

Inflammatory bowel diseases are intractable inflammatory diseases of the large and small intestines caused by a variety of factors. In the inflammatory bowel diseases, the small number of patients have affected regions around the rectum, and the majority of patients have those extends over a wide range to the descending colon or transverse colon. Accordingly, administration using suppositories cannot be expected to produce desired therapeutic effects in the large number of patients with inflammatory bowel diseases.

On the other hand, in administration using enema preparations, high therapeutic effects can be expected because the active ingredients can directly reach to the affected regions. However, when the current marketed drugs described above are administered for treating regional enteritis or ulcerative colitis in the dosage form of enema preparations, the improvement in therapeutic effects is only 1.2 to 2.3 times as potent as those of the oral preparations or injections.

### DISCLOSURE OF THE INVENTION

The object of this invention is to provide a drug, which can provide much higher therapeutic effects to patients with inflammatory bowel diseases.

The present inventors have directed tremendous research effort toward the development of pharmacologically active ingredients that provide further significantly improved therapeutic effects when administered in the form of an enema preparation than when administered in the form of an oral preparation. As a result, the inventors have finally found a surprising fact, which even those skills in the art could not predict, that the use of thiazole compounds having the following general formula (1) or the salts thereof as a pharmacologically active ingredient makes it possible to significantly improve their therapeutic effects when they are administered in the form of an enema preparation than when they are administered in the form of an oral preparation. This invention has been made based on such findings.

1. This invention is an enema preparation that contains at least one selected from the group consisting of thiazole compounds having the following general formula and the salts thereof: 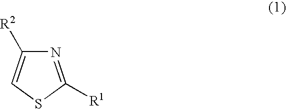
wherein R¹ represents a phenyl group which may have 1 to 3 lower alkoxy groups as substituents on the phenyl ring and R² a pyridyl group which may have 1 to 3 carboxyl groups as substituents on the pyridine ring.

2. This invention is the enema preparation of the above description 1, wherein the thiazole compound is 6-[2-(3,4-diethoxyphenyl)thiazol-4-yl]pyridine-2-carboxylic acid.

3. This invention is the enema preparation of the above description 1 or 2 used in the treatment of inflammatory bowel diseases.

4. This invention is an enema preparation for use in the treatment of inflammatory bowel diseases that contains at least one selected from the group consisting of thiazole compounds having the general formula (1) and the salts thereof.

The thiazole compounds of the general formula (1) used in this invention are known compounds and can be produced, for example, by the method described in Japanese Patent Laid-Open No. 5-51318 (JP-A-5-51318).

Specifically, the groups shown in the general formula (1) described above are as follows.

Phenyl groups which may have 1 to 3 lower alkoxy groups, as substituents, on the phenyl rings include those which may have 1 to 3 straight-chain or branched-chain alkoxy groups with 1 to 6 carbon atoms, as substituents, on the phenyl rings, such as phenyl, 2-methoxyphenyl, 3-methoxyphenyl, 4-methoxyphenyl, 2-ethoxyphenyl, 3-ethoxyphenyl, 4-ethoxyphenyl, 4-isopropoxyphenyl, 4-pentyloxyphenyl, 3-ethoxy-4-methoxyphenyl, 4-hexyloxyphenyl, 3,4-dimethoxyphenyl, 3,4-diethoxyphenyl, 2,3-dimethoxyphenyl, 2,6-dimethoxyphenyl, 3-propoxy-4-methoxyphenyl, 3,5-dimethoxyphenyl, 3,4-dipentyloxyphenyl, 3,4,5-trimethoxyphenyl and 3-methoxy-4-ethoxyphenyl.

Pyridyl groups which may have 1 to 3 carboxyl groups as substituents on the pyridine rings include, for example, pyridyl, 2-carboxylpyridyl, 3-carboxylpyridyl, 4-carboxylpyridyl, 2,3-dicarboxylpyridyl, 3,4-dicarboxylpyridyl, 2,4-dicarboxylpyridyl, 3,5-dicarboxylpyridyl, 3,6-dicarboxylpyridyl, 2,6-dicarboxylpyridyl and 2,4,6-tricarboxylpyridyl.

Of the thiazole compounds represented by the general formula (I) of the present invention, those having basic groups can easily form salts with ordinary pharmacologically acceptable acids. Examples of such acids are: inorganic acids such as sulfuric acid, nitric acid, hydrochloric acid, phosphoric acid and hydrobromic acid; and organic acids such as acetic acid, p-toluenesulfonic acid, ethanesulfonic acid, oxalic acid, maleic acid, fumaric acid, malic acid, tartaric acid, citric acid, succinic acid and benzoic acid.

Of the thiazole compounds represented by the general formula (I) of the present invention, those having acidic groups can easily form salts with pharmacologically acceptable basic compounds. Examples of such basic compounds are sodium hydroxide, potassium hydroxide, calcium hydroxide, sodium carbonate and potassium hydrogencarbonate.

The thiazole compounds of this invention include optical isomers.

The enema preparation of this invention contains one or two or more thiazole compounds having the general formula (1) and the salts thereof as pharmacologically active ingredients.

The enema preparation of this invention may take any one of the solution, suspension, zol and gel forms.

The solution form of the enema preparation of this invention is prepared by dissolving a pharmacologically active ingredient in an aqueous solvent or nonaqueous solvent. Such aqueous solvents include, for example, water. Such nonaqueous solvents include, for example, glycerol, ethylene glycol, propylene glycol, polyethylene glycol, polypropylene glycol and vegetable oils.

The enema preparation of this invention may contain known excipients, such as thickener, buffer, preservative and pH adjustor, as need arises.

Such thickeners include, for example, sodium carboxymethylcellulose and carboxyvinyl polymer.

Such buffers include, for example, sodium hydrogenphosphate, sodium acetate and tris(hydroxymethyl)aminomethane.

Such preservatives include, for example, sodium edentate, ethyl paraoxybenzoate, butyl paraoxybenzoate and sodium pyrosulfite.

Such pH adjustors include, for example, sodium hydroxide and hydrochloric acid.

The amount of the pharmacologically active ingredient contained in the enema preparation of this invention is not limited to any specific one, but properly selected from those ranging widely. Generally, its content in the enema preparation is about 0.001 to 70% by weight preferably.

The enema preparation of this invention is administered by known methods for example, it is administered rectally.

The amount of the enema preparation of this invention administered is properly selected according to its usage, the age, sex and other conditions of patients, and the severity of diseases. Generally, the enema preparation is administered so that the amount of the pharmacologically active ingredient administered is about 0.02 to 2000 mg/kg body weight/day preferably.

The enema preparation of this invention is suitably used for the treatment of inflammatory bowel diseases. Inflammatory bowel diseases are intractable inflammatory diseases of the large and small intestines caused by various factors as described above. Inflammatory bowel diseases include, for example, ulcerative colitis as a diffuse nonspecific inflammation of uncertain factors which affect the mucous membrane of the colon and form erosion or ulceration of the colon; Crohn's disease, a nonspecific granulomatous inflammatory diseases of uncertain factors which result in fibrosis or ulceration of the colon; a pathological change of the intestinal tract in Behcet's disease, as a chronic systemic inflammatory disease; hemorrhagic rectal ulcer; ileal pouchitis; intestinal tuberculosis; ischemic colitis; drug-induced colitis; radiation-induced colitis; and infective colitis.

The symptoms associated with inflammatory bowel diseases include, for example, abdominal pain, general malaise, diarrhea, melena, positive occult blood, fever, anorexia, weight loss, anemia, ileus, abdominal mass, nausea, vomiting, symptoms of peritonitis and mucous stool.

The use of the enema preparation of this invention shows the therapeutic actions against inflammatory bowel diseases to be about 10 times more potent than that obtained in oral form of the same formulation, consequently the amount of the preparation used can be significantly decreased, resulting in considerable suppression of the onset of adverse effects.

The Use of the enema preparation of this invention enables the pharmacologically active ingredient to rapidly reach the affected region needing treatment, consequently the preparation can develop therapeutic effects with rapid action.

### BEST MODE FOR CARRYING OUT THE INVENTION

In the followings, this invention will be described in more detail taking an example and a pharmacological test example.

### EXAMPLE 1

| | |
|---|---|
| Pharmacologically active ingredient | 1.0 mg/ml |
| Tris(hydroxymethyl)aminomethane | 1.2 mg/ml |
| Sodium hydroxide | 1.2 mg/ml |
| Sodium carboxymethylcellulose | 15 mg/ml |
| Hydrochloric acid | adequate amount |
| Purified water | adequate amount |

Tris(hydroxymethyl)aminomethane, sodium hydroxide (solubilizing agent) and hydrochloric acid (pH adjustor) were dissolved in purified water to prepare a solution with pH 9.3. Then, a pharmacologically active ingredient (6-[2-(3,4-diethoxyphenyl)thiazol-4-yl]pyridine-2-carboxylic acid) and sodium carboxymethylcellulose were dissolved in the solution to prepare an enema preparation.

### PHARMACOLOGICAL TEST EXAMPLE

(1) Preparation of a Rat Model of Colitis

A rat model of colitis was prepared in accordance with the method by Morris et al. (Morris GP et al., Hapten-induced model of chronic inflammation and ulceration in the rat colon. Gasteroenterology, 96: 795-803, 1989). Specifically, the rats were fasted for approximately 24 hr and anesthetized with ether. A Teflon catheter (registered trade mark) was inserted into the lumen of the colon via the anus. The tip of the catheter was advanced to 8 cm from the anus, and 0.25 ml of TNBS solution (final concentration of 60 mg/mL in 50% ethanol) was injected into the colon. Rats were held in a vertical position for 30 seconds after TNBS injection (Day-0).

One day after an injection of TNBS (Day-1), rats were randomly allocated to the four groups described below based on body weight using a stratified random sampling method. The groups were as follows:

Group A: a vehicle control group

Group B: a group administered with the pharmacologically active ingredient in an amount of 0.1 mg/kg/day

Group C: a group administered with the pharmacologically active ingredient in an amount of 0.3 mg/kg/day

Group D: a group administered with the pharmacologically active ingredient in an amount of 1 mg/kg/day

(2) Administration of Pharmacologically Active Ingredient

The pharmacologically active ingredient and its vehicle were delivered at 8 cm orad to the anal verge of conscious rats through a Teflon catheter (registered trade mark) inserted into the lumen of the colon via the anus. They were administered once daily in the morning for 7 consecutive days from Day-1.

(3) Evaluation of Diarrhea

On the Day-B, the rats were anesthetized with ether, and sacrificed by exsanguination. The colon, from the end of the cecum to the anus, was excised, opened by a longitudinal incision, and the feces were macroscopically assessed at the excision of colons. When feces were not solid, it ws judged as diarrhea. Rats with no feces in the colon were excluded from diarrhea evaluation.

(4) Statistical Analysis

The results of the incidence of diarrhea were expressed as the proportion of rats with diarrhea in each group. Statistical analysis was performed between the vehicle control group (Group A) and the groups administered with the pharmacologically active ingredient (Group B to D) using Fisher's exact test (two-sided) to which Bonferroni correction, which takes multiplicity into consideration, was applied. Statistical significance is defined as p<0.05.

(5) Results

The incidences of diarrhea in Groups A, B, C and D were 73.9% ( 17/23), 33.3% ( 7/21, p=0.0432), 20.0% ( 5/25, p=0.0011) and 20.0% ( 4/20, p=0.0020), respectively. In all the groups, which were administered with the pharmacologically active ingredient (Groups B to D), the incidence of diarrhea could be suppressed significantly, compared with the vehicle control group (Group A).

This result revealed that the once-daily enema administration of the pharmacologically active ingredient in amounts of 0.1 to 1 mg/kg/day beginning the day after the onset of colic disorder for 7 consecutive days can significantly suppress the incidence of diarrhea, which is one of the important indices of assessment in a rat model of colitis.

Further, the incidence of diarrhea with oral administration of the pharmacologically active ingredient described above in the same rat model of colitis showed that 1 mg/kg/day of the pharmacologically active ingredient was significantly effective; however, no significant decrease in the incidence of diarrhea was observed when the amount of the pharmacologically active ingredient administered was lower than 1 mg/kg/day. This revealed that when the above pharmacologically active ingredient was intra-rectally administered, the significant disorder-suppressing effect was observed in amounts one tenth as much as those with orally administration.

1. An enema preparation, comprising at least one selected from the group consisting of thiazole compounds having the following general formula and the salts thereof: 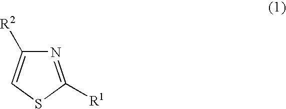
wherein R¹ represents a phenyl group which may have 1 to 3 lower alkoxy groups as substituents on the phenyl ring and R² a pyridyl group which may have 1 to 3 carboxyl groups as substituents on the pyridine ring.

2. The enema preparation according to claim 1, wherein the thiazole compound is 6-(2-(3,4-diethoxyphenyl)thiazol-4-yl]pyridine-2-carboxylic acid.

3. The enema preparation according to claim 1 or 2, used in the treatment of inflammatory bowel diseases.

4. A method for treating inflammatory bowel diseases, by administering a patient in need thereof, an enema preparation, comprising at least one selected from the group consisting of thiazole compounds having the following general formula and the salts thereof: 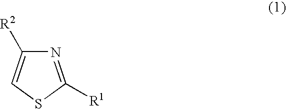
wherein R¹ represents a phenyl group which may have 1 to 3 lower alkoxy groups as substituents on the phenyl ring and R² a pyridyl group which may have 1 to 3 carboxyl groups as substituents on the pyridine ring.

5. The method according to claim 4, wherein the thiazole compound is 6-[2-(3,4-diethoxyphenyl)thiazol-4-yl]pyridine-2-carboxylic acid.

6. A use of a compound for the production of a medicament for treating inflammatory bowel diseases, which medicament is an enema preparation, comprising at least one selected from the group consisting of thiazole compounds having the following general formula and the salts thereof: 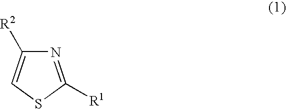
wherein R¹ represents a phenyl group which may have 1 to 3 lower alkoxy groups as substituents on the phenyl ring and R² a pyridyl group which may have 1 to 3 carboxyl groups as substituents on the pyridine ring.

7. The use according to claim 6, wherein the thiazole compound is 6-[2-(3,4-diethoxyphenyl)thiazol-4-yl]pyridine-2-carboxylic acid.
